(19) United States
(12) Patent Application Publication
(10) Pub. No.: US 2026/0209674 A1
(43) Pub. Date: Jul. 23, 2026

(54) COMPRESSIBLE BIOREATOR CONTAINER WITH GAS OUTLET COMPRISING A FLOW RESTRICTOR AND METHOD OF MIXING A CELL SUSPENSION THEREIN

(71) Applicant: Oribiotech Ltd, London, Greater London (GB)

(72) Inventors: Arman Amini, London (GB); Derek Bean, Northamptonshire (GB); Stuart Milne, Cambridge (GB); Shaun Mansfield, London (GB); Zakariyah Karimijee, Cambridge (GB); Matthew Williams, Cambridge (GB); Thomas Edward Parker, Cambridge (GB); Jason Palmer, Cambridge (GB); Martin Hoole, Cambridge (GB); Christopher Rosier, Cambridge (GB); John Stephenson, Cambridge (GB)

(21) Appl. No.: 19/141,150

(22) PCT Filed: Dec. 19, 2023
(86) PCT No.: PCT/GB2023/053304

§ 371 Date: Jun. 19, 2025

(30) Foreign Application Priority Data
Dec. 20, 2022 (GB) 2219300.7
Jul. 25, 2023 (GB) 2311394.7

(51) Int. Cl. C12M 1/00; B01F 23/53; B01F 31/31; B01F 35/00; B01F 101/44; C12M 1/02; C12M 1/12; C12M 1/26

(52) U.S. Cl. CPC C12M 29/20; B01F 23/53; B01F 31/31; B01F 35/187; C12M 23/06; C12M 23/26; C12M 27/00; C12M 29/04; C12M 33/00; B01F 2101/44; B01F 2215/0431

(57) ABSTRACT

A bioreactor and methods of using a bioreactor are disclosed. The bioreactor includes a compressible bioreactor container configured to hold a cell suspension in an internal volume of the compressible bioreactor container. The compressible bioreactor container includes a gas outlet in fluid communication with the internal volume of the compressible bioreactor container and configured to permit gas flow out of the internal volume of the compressible bioreactor container in use. The gas outlet includes a flow restrictor configured, such that when the compressible bioreactor container is compressed in use, the flow restrictor restricts gas flow out of the gas outlet.

### CROSS-REFERENCE TO RELATED APPLICATIONS

This application is a national phase entry under 35 U.S.C. § 371 of International Patent Application PCT/GB2023/053304, filed Dec. 19, 2023, designating the United States of America and published as International Patent Publication WO 2024/134179 A1 on Jun. 27, 2024, which claims the benefit under Article 8 of the Patent Cooperation Treaty of United Kingdom Patent Application Serial No. 2219300.7, filed Dec. 20, 2022, and of United Kingdom Patent Application Serial No. 2311394.7, filed Jul. 25, 2023.

### TECHNICAL FIELD

The disclosure relates to a bioreactor having a compressible bioreactor container, and, in particular, the bioreactor having a compressible bioreactor container with a flow restrictor to restrict gas flow out of an outlet of the bioreactor container.

### BACKGROUND

Biological handling processes, such as cell and gene therapy (CGT) manufacturing processes, are often complex and include manual steps across several devices. Equipment systems used in various steps or unit operations, of cell-based therapeutic products (CTP) manufacturing may include devices for various unit operations. The unit operations may include, for example, cell collection, cell isolation, selection, cell expansion, cell washing, volume reduction, cell storage or transportation. The unit operations can vary immensely based on the manufacturing model (i.e., autologous versus allogenic), cell type, intended purpose, among other factors. In addition, cells are “living” entities sensitive to even the simplest manipulations (such as differences in a cell transferring procedure). The role of cell manufacturing equipment in ensuring scalability and reproducibility is an important factor for cell and gene therapy manufacturing.

In addition, cell-based therapeutic products (CTP) have gained significant momentum thus there is a need for improved cell manufacturing equipment for various cell manufacturing procedures, for example, but not limited to stem cell enrichment, generation of chimeric antigen receptor (CAR) T cells, and various cell manufacturing processes such as collection, purification, gene modification, incubation/recovery, washing, infusion into patient and/or freezing.

The culture or processing of cells typically requires the use of a device to hold the cells, for example, in an appropriate culture medium when culturing the cells. The known devices include shaker flasks, roller bottles, T-flasks and bags. Such bottles or flasks are widely used but suffer from several drawbacks. During cell culturing further culturing media may be added to the container, and some fluid may be extracted from the container, for example, a waste fluid.

### BRIEF SUMMARY

In accordance with one aspect of the present disclosure, there is provided a bioreactor comprising a compressible bioreactor container configured to hold a cell suspension in an internal volume of the compressible bioreactor container. The compressible bioreactor container comprises a gas outlet in fluid communication with the internal volume of the compressible bioreactor container and configured to permit gas flow out of the internal volume of the compressible bioreactor container in use. The gas outlet comprises a flow restrictor configured such that when the compressible bioreactor container is compressed in use the flow restrictor restricts gas flow out of the gas outlet.

The flow restrictor restricts gas flow out of the gas outlet to reduce the flow rate of gas flow out of the bioreactor during compression. This permits pressure to build up in the bioreactor, and the compressible bioreactor container can expand to compensate for the pressure build up. The reduced flow rate out of the bioreactor also minimizes the loss of water vapor from the bioreactor container through the outlet. It is beneficial to keep water vapor (and other gaseous components) within the bioreactor container, in order to maintain the optimum internal environment for cell growth, and the flow restrictor improves this during compression of the bioreactor container.

In examples, the bioreactor may further comprise an expandable expansion container having an internal volume fluidly connected to the internal volume of the compressible bioreactor container. In this way, when the compressible bioreactor container is compressed in use, gas in the internal volume of the compressible bioreactor container is displaced into the internal volume of the expansion container. This increases the gas pressure in the expansion container and the expansion container expands to compensate for this increase in gas pressure. The expansion container also permits expansion and contraction of the compressible bioreactor container without greatly changing the pressure in the compressible bioreactor container. This is due to the displacement of gases between the compressible bioreactor container and the expansion container as the compressible bioreactor container is compressed and expanded.

In examples, the expansion container may comprise the gas outlet. Accordingly, as the pressure in the expansion container increases, gas will flow out of the expansion container through the gas outlet. The flow restrictor will restrict gas flow out of the gas outlet. The restricted flow of gas out of the gas outlet permits pressure in the expansion container to increase, and the expansion container will expand to compensate for this increase in gas pressure. Accordingly, less gas egresses through the gas outlet and the expansion container accommodates more gas due to the expansion under pressure. Gas retained in the expansion container can return to the bioreactor container, for example, after compression is reversed.

In examples, the compressible bioreactor container may comprise a base section, a top section, and a compressible side wall extending between the base section and the top section. Accordingly, the compressible bioreactor container can expand and contract to compensate for the pressure therein. The compressible bioreactor container can also be actuated to expand and contract to control the internal volume of the compressible bioreactor container, or to agitate or mix the cell suspension contained in the internal volume of the compressible bioreactor container.

In examples, the expansion container may be connected to the top section of the compressible bioreactor container. Accordingly, expansion of the expandable container is not restricted by any external surfaces.

In examples, the top section of the compressible bioreactor container may comprise an interface plate having an opening. The expansion container may be mounted to the interface plate at the opening such that the internal volume of the expansion container is fluidly connected to the internal volume of the compressible bioreactor container via the opening in the interface plate.

In examples, the expansion container may comprise a base section, a top section, and a compressible side wall extending between the base section and the top section. The base section may be attached to the interface plate. Accordingly, the expansion container can expand and contract to compensate for the pressure therein. The expansion container can also be actuated to expand and contract to control the volume of the expansion container.

In examples, the compressible bioreactor container and/or the expansion container may be generally cylindrical, where the side wall is generally cylindrical. The bioreactor interface plate may be generally circular and planar.

In examples, the side wall of the compressible bioreactor container and/or the expansion container may be a bellows wall. The side wall may include a plurality of inward folds and outward folds, interleaved with leaf segments. The leaf segments may be rigid. The inward and outward folds permit the leaf segments to fold against each other, thereby compressing the side wall, or vice versa to extend the side wall.

In examples, the bioreactor container may further comprise one or more ports for introducing material into and/or removing material from an internal volume of the compressible bioreactor container. In examples, the one or more ports may be provided in the interface plate. The one or more ports may comprise a seal, for example, a septum seal. Accordingly, material can be introduced into, or extracted from the internal volume of the compressible bioreactor container through the ports. The seals cover and seal the ports to prevent the introduction of contaminants into the internal volume of the compressible bioreactor container and maintain an aseptic environment within the internal volume.

In examples, the flow restrictor comprises a valve. In some examples, the valve may permit gas flow into and/or out of the gas outlet when a differential gas pressure at the gas outlet is at or above a threshold differential pressure. Accordingly, gas is blocked from flowing out of the gas outlet when the differential gas pressure at the gas outlet is below a threshold differential pressure, preventing the loss of water vapor from the bioreactor container through the outlet. Furthermore, gas is permitted to flow into and/or out of the gas outlet when the differential gas pressure at the gas outlet is at or above the threshold differential pressure. This advantageously prevents substantial fluctuations in pressure in the bioreactor container during expansion and compression of the bioreactor container and also reduces the force required to expand and compress the bioreactor container.

In examples, the valve may not restrict the flow rate of gas therethrough when the differential gas pressure at the gas outlet is at or above the threshold differential pressure. Accordingly, this reduces the force required to expand and/or compress the bioreactor container.

In examples, the valve may be a two-way valve. In some examples, the valve may comprise a combination of an umbrella valve and a duckbill valve. In other examples, the valve may be a ball valve, in particular, a two-way ball valve. In other examples, the valve may be an elastic constriction valve. In other examples, the valve may be a membrane valve having a membrane with one or more slits.

Accordingly, gas flow is permitted into and out of the gas outlet during expansion and compression of the bioreactor container in order to compensate for the change of pressure in the bioreactor container due to the change in volume thereof during expansion and compression.

In examples, the flow restrictor may comprise a flow restriction path having a restricted diameter. Accordingly, the flow restriction path having the restricted diameter reduces the flow rate of gas through the gas outlet, and so pressure within the bioreactor container increases as the bioreactor container is compressed, causing expansion of the bioreactor container.

In examples, the restricted diameter may be between about 0.15 mm and about 1.5 mm. The restricted diameter may be between about 0.5 mm and about 1.0 mm. The restricted diameter may be one of one of about 0.5 mm, about 0.75 mm, or about 1.0 mm.

In examples, the gas outlet may have an outlet diameter, and the restricted diameter may be smaller than the outlet diameter. Accordingly, the diameter of the gas outlet is modified to provide a restricted diameter through which gas can flow through the gas outlet.

In examples, the flow restrictor may comprise a porous structure arranged to restrict the gas flow out of the outlet. The porous structure may be a sintered material. The porous structure may be a filter. The porous structure may restrict the flow of gases therethrough to reduce the flow rate of gas through the outlet.

In examples, the flow restrictor may be disposed in the gas outlet. The flow restrictor may be press-fit into the gas outlet. In some examples, the flow restrictor may be disposed over the gas outlet. The flow restrictor may be press-fit onto an outer surface of the gas outlet. In other examples, the flow restrictor may be integrally formed with the gas outlet.

In examples, the flow restrictor may be integrally formed with the gas outlet.

In examples, the flow restrictor may comprise a tube fluidly connected to the gas outlet. Accordingly, as gas flows out of the gas outlet into the tube, the pressure at the gas outlet increases, thereby restricting gas flow out of the gas outlet.

In examples, an internal diameter of the tube may be between about 0.2 mm and about 1 mm. The internal diameter of the tube may be about 0.5 mm.

In examples, a length of the tube may be at least about 100 mm. The length of the tube may be between about 100 mm and about 700 mm. The length of the tube may be between about 300 mm and about 500 mm. The length of the tube may be one of about 300 mm or about 500 mm.

In examples, the flow restrictor may comprise a user-actuatable valve. Actuation of the user-actuatable valve may be independent of a differential gas pressure across the valve.

The user-actuatable valve may include a spring element operably coupled to a moveable plate. The movable plate may be operable to translate to selectively block an inlet or outlet of the valve.

The spring element may be arranged to bias the movable plate into either a closed position or an open position. The spring element may have a resilience selected so that differential pressure fluctuations during use of the bioreactor do not inadvertently switch the movable plate between the closed position and the open position.

The user-actuatable valve may also comprise a valve actuator configured to switch the movable plate from the closed valve position to the open valve position and/or vice versa.

The valve actuator may comprise a control arm operably connected to a controller. In alternate examples, the user-actuatable valve may be actuated by any other suitable valve actuator. For example, the user-actuatable valve may form a twist valve, an electrically-actuated valve (e.g., a solenoid valve) or a magnetically actuated valve.

The user-actuatable valve may be configured such that switching between the open and closed valve position is independent of the pressure differential across the valve.

The user-actuatable valve may be in the closed valve position during a compression mixing operation. The user-actuatable valve may be open valve position during a breathing operation to permit maximum air exchange with the contents of the bioreactor container and/or the expansion container.

The controller may be configured to actuate the valve in accordance with a pre-defined operating mode sequence.

In examples, the bioreactor may further comprise a filter provided in a gas flow path between the internal volume of the compressible bioreactor container and the gas outlet and/or the flow restrictor. The filter may be provided in the gas outlet. The filter may comprise a hollow body that provides a channel through the filter, and a filter element extending across the channel. The hollow body may be wider at a central portion of the hollow body and the filter element may be arranged within the central portion. The hollow body may have an end portion extending from each end of the central portion. One or both of the end portions may comprise the flow restrictor. Each end portion may be formed as a spigot. The filter element may be a polyethersulfone membrane or a polyvinylidene difluoride membrane. Accordingly, the filter can remove contaminants from gas flowing therethrough. This prevents contaminants in the ambient environment from reaching the cell suspension in the internal volume of the compressible bioreactor container, thereby preventing contamination of the cell suspension.

In accordance with a further aspect of the present disclosure, there is provided a filter for a gas outlet of a bioreactor. The filter may comprise a hollow body that provides a channel. The hollow body comprises a central portion and an end portion extending from the central portion. The central portion is wider than the end portion. A filter element is arranged within the central portion so as to extend across the channel. The end portion has a reduced diameter to restrict airflow through the channel.

In this way, the filter may be used in a gas outlet of a bioreactor container. The reduced diameter of the end portion restricts gas flow out of the gas outlet to reduce the flow rate through the filter.

In examples, the end portion is a first end portion and the filter may comprise a second end portion extending from an opposite side of the central portion to the first end portion.

In examples, a flow restrictor may be provided in the first end portion and/or the second end portion to reduce the diameter and restrict airflow through the channel.

In examples, the flow restrictor may be press-fit into the first end portion and/or the second end portion. In other examples, the flow restrictor may be integrally formed with the first end portion and/or the second end portion.

In examples, the flow restrictor may be integrally formed with the first end portion and/or the second end portion.

In examples, the first end portion and/or the second end portion may be formed as a spigot.

In examples, the filter element may be a polyethersulfone membrane or a polyvinylidene difluoride membrane.

In accordance with a further aspect of the present disclosure, there is provided a method of mixing a cell suspension in a bioreactor. In examples, the method is a method of mixing a cell suspension using the bioreactor container described above.

The method comprises providing a bioreactor comprising a compressible bioreactor container with a gas outlet comprising a flow restrictor, introducing a cell suspension to an internal volume of the compressible bioreactor container, compressing the compressible bioreactor container to reduce the internal volume of the compressible bioreactor container, and while compressing the compressible bioreactor container, restricting gas flow through the gas outlet by the flow restrictor.

In examples, the compressible bioreactor container may comprise an expansion container having an internal volume fluidly connected to the internal volume of the compressible bioreactor container. The method may comprise displacing gas from the internal volume of the compressible bioreactor container into the internal volume of the expansion container during compression of the compressible bioreactor container.

In examples, the flow restrictor may comprise a valve. The restricting the gas flow through the gas outlet by the flow restrictor may comprise blocking gas flow through the valve when a differential gas pressure at the gas outlet is below a threshold differential pressure and permitting gas flow through the valve when the differential gas pressure at the gas outlet is at or above the threshold differential pressure.

### BRIEF DESCRIPTION OF THE DRAWINGS

Examples of the invention are now described, by way of example only, hereinafter with reference to the accompanying drawings, in which:

FIGS. 1A and 1B illustrate a bioreactor;

FIG. 2 illustrates a cross-sectional view of the bioreactor of FIG. 1;

FIG. 3 illustrates a cross-section perspective view of the bioreactor of FIG. 1;

FIG. 4 illustrates a cross-sectional view of an outlet of the bioreactor of FIG. 1 with a first example of a flow restrictor;

FIGS. 5A and 5B illustrate arrangements of the flow restrictor of FIG. 4;

FIG. 6 illustrates a second example of a flow restrictor;

FIGS. 7A and 7B illustrate the outlet of the bioreactor of FIG. 1 with a third example of a flow restrictor;

FIGS. 8A to 8D illustrate a fourth example of a flow restrictor;

FIGS. 9A and 9B illustrate a fifth example of a flow restrictor;

FIGS. 10A to 10C illustrate a sixth example of a flow restrictor;

FIGS. 11A and 11B illustrate a seventh example of a flow restrictor; and

FIG. 12 illustrates a graph of the load required to compress the bioreactor container with different flow restrictors.

### DETAILED DESCRIPTION

The described example embodiments relate to an assembly for handling biological material. In particular, some embodiments relate to an assembly that is aseptic, or sterile. It is noted that the terms “aseptic” and “sterile” may be used interchangeably throughout the present disclosure. References to fluids in the detailed description are not intended to limit the scope of protection to such materials. As will be recognized by a person skilled in the art, fluids as described herein are merely an example of a suitable material for use with the assembly as described. Equally, reference may be made to a container, container, or the like, however, such references are not intended to limit the scope of protection to such containers or containers. As will be recognized by a person skilled in the art, containers, containers or the like are described herein as mere examples.

Certain terminology is used in the following description for convenience only and is not limiting. The words ‘upper’ and ‘lower’ designate directions in the drawings to which reference is made and are with respect to the described component when assembled and mounted. The words ‘inner,’ ‘inwardly’ and ‘outer,’ and ‘outwardly’ refer to directions toward and away from, respectively, a designated centerline or a geometric center of an element being described (e.g., a central axis), the particular meaning being readily apparent from the context of the description. Further, the terms ‘proximal’ (i.e., nearer to) and ‘distal’ (i.e., away from) designate positions relative to an axis or a point of attachment.

Further, as used herein, the terms ‘connected,’ ‘affixed,’ ‘coupled’ and the like are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, ‘first,’ ‘second,’ ‘third’ etc., merely indicate that different instances of like objects are being referred to and are not intended to imply that the objects so described must be in a given sequence, either temporally, spatially, in ranking or in any other manner. Like reference numerals are used to depict like features throughout.

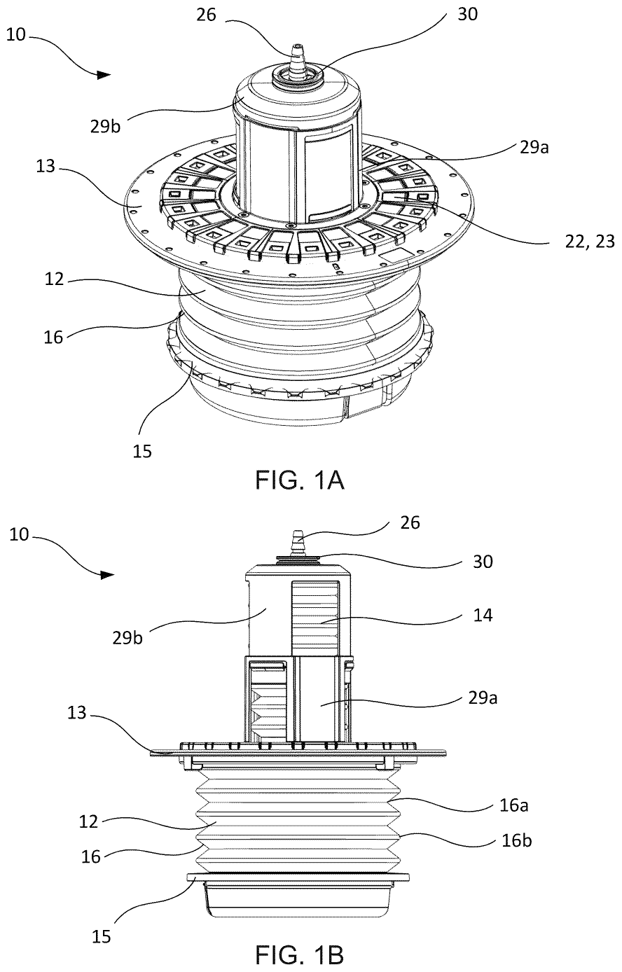

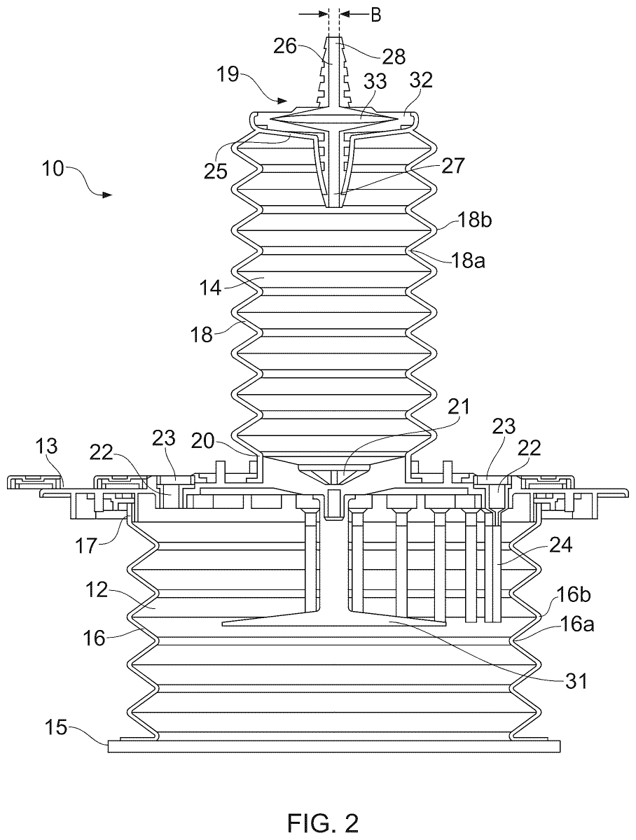

FIGS. 1A, 1B and 2 show a bioreactor 10 of the present disclosure. The bioreactor 10 includes a compressible bioreactor container 12, an interface plate 13 and an expansion container 14, otherwise called a breathing container.

During use, the compressible bioreactor container 12 has an internal volume that holds a fluid in which cell processing occurs. In particular, the fluid is a cell suspension and comprises a population of cells present in a liquid medium. In various examples, the population of cells provided to the compressible bioreactor container 12 in use may comprise any human or animal cell type, for example: any type of adult stem cell or primary cell, T cells, CAR-T cells, monocytes, leukocytes, erythrocytes, NK cells, gamma delta t cells, tumor infiltrating t cells, mesenchymal stem cells, embryonic stem cells, induced pluripotent stem cells, adipose derived stem cells, Chinese hamster ovary cells, NS0 mouse myeloma cells, HELA cells, fibroblasts, HEK cells, insect cells, organoids etc. Suitably the population of cells may comprise T-cells. Alternatively, the population of cells may comprise any microorganism cell type, for example: bacterial, fungal, Archaean, protozoan, algal cells.

The compressible bioreactor container 12 and the expansion container 14 are compressible, for example, by having a bellows wall. The compressible bioreactor container 12 can expand and retract as it is filled and emptied. The expansion container 14 can expand and retract as gas is transferred into and out of the expansion container 14 from the compressible bioreactor container 12.

As shown in FIGS. 1A, 1B and 2, the compressible bioreactor container 12 has a base section having a bottom wall 15, a top section having the interface plate 13, and a compressible side wall 16. The bottom wall 15 is disposed opposite the interface plate 13. The bottom wall 15 is rigid, or attached to a rigid plate, so as to provide a bottom surface of the compressible bioreactor container 12. A top part 17 of the compressible side wall 16 is attached to the interface plate 13, as shown in FIG. 2. The top part 17 may include a rigid ring or similar for attaching to the interface plate 13. The compressible side wall 16 is compressible such that the bottom wall 15 can move toward and away from the interface plate 13, varying the internal volume of the compressible bioreactor container 12.

The compressible side wall 16 may be a bellows wall, having a concertina arrangement that allows the compressible side wall 16 to fold onto itself in order to compress. In particular, the compressible side wall 16 may comprise a series of alternately arranged inward folds 16a and outward folds 16b that allow the compressible side wall 16 to compress like a bellows or concertina. Rigid leaf portions extend between the inward folds 16a and the outward folds 16b. The inward folds 16a and outward folds 16b may be formed by thinned sections in the compressible side wall 16, with the inward folds 16a having a thinned section arranged on the outer surface of the compressible side wall 16, and the outward folds 16b having a thinned section arranged on the inner surface of the compressible side wall 16.

The compressible bioreactor container 12 can therefore expand and contract, or be expanded and contracted, according to the material held in the compressible bioreactor container 12. In particular, the compressible bioreactor container 12 may expand as the cell culture within the compressible bioreactor container 12 grows, and/or as additional materials are added. The compressible bioreactor container 12 may be contracted and expanded by an actuator (not shown) adapted to move, for example, push and/or pull, the bottom wall 15 of the compressible bioreactor container 12 and/or the interface plate 13 to change the volume of the compressible bioreactor container 12.

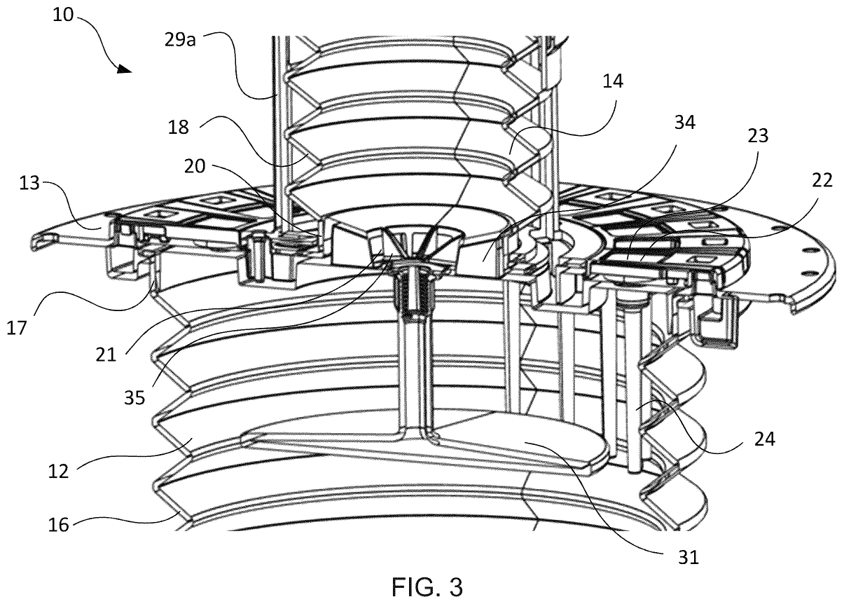

As shown in FIGS. 2 and 3, the interface plate 13 has a lower surface that is sealingly connected to the top part 17 of the compressible side wall 16 of the compressible bioreactor container 12. The interface plate 13 also has an upper surface that is sealingly connected to a lower part 20 of a compressible side wall 18 of the expansion container 14.

The interface plate 13 has one or more ports 22 for transfer of material into and out of the internal volume compressible bioreactor container 12. An external component can be connected to one or more of the ports 22 to introduce material through the ports 22. Each of the ports includes a seal 23, for example, a septum seal, that maintains a sealed environment within the internal volume of the compressible bioreactor container 12 and also permits a needle to pass through to create a fluid connection into the internal volume of the compressible bioreactor container 12. In alternative examples, each port 22 may have a valve, a cap, or other closure that provides an openable or breakable seal. One or more of the ports 22 may have a dip tube 24 that extends from the port 22 into the internal volume of the compressible bioreactor container 12. The dip tubes 24 can extend into the cell suspension in use, so as to be used to remove material, for example, a sample, from the compressible bioreactor container 12.

A baffle 31 is mounted to the interface plate 13 such that the baffle 31 is suspended within the internal volume of the compressible bioreactor container 12. The baffle 31 may be attachable to the interface plate 13 by a threaded connector, or by a clip or clamp. The baffle 31 is attached to the center of the interface plate 13 such that the baffle 31 is centrally positioned within the compressible bioreactor container 12. However, it will be appreciated that the baffle 31 may be positioned off-center within the compressible bioreactor container 12. The bottom surface of the baffle 31 is substantially flat and faces the bottom wall 15 of the compressible bioreactor container 12. The baffle 31 also has a conical upper surface facing the interface plate 13. The baffle 31 is circular and is sized so as to be spaced from the compressible side wall 16 of the compressible bioreactor container 12. This allows the dip tubes 24 to pass between the compressible side wall 16 and the baffle 31 so as to provide a fluid sampling path from the compressible bioreactor container 12 to the interface plate 13.

The baffle 31 is provided to mix the cell suspension contained in the internal volume of the compressible bioreactor container 12 during use. In particular, the bottom wall 15 of the compressible bioreactor container 12 may be moved relative to the interface plate 13 and baffle 31 such that the baffle 31 contacts the cell suspension within the internal volume of the compressible bioreactor container 12 and mixes it. In examples, the bottom wall 15 may be raised and lowered relative to the interface plate 13 (i.e., to change a distance between the bottom wall 15 and the interface plate 13), and/or the bottom wall 15 may be tilted relative to the interface plate 13, and/or the bottom wall 15 may be rotated relative to the interface plate 13.

The interface plate 13 has an aperture 21 that extends between the upper surface and the lower surface of the interface plate 13 and permits gas flow between the internal volume of the compressible bioreactor container 12 and the internal volume of the expansion container 14. The aperture 21 may include a filter. The filter may prevent particulates, such as cells, from transferring from the compressible bioreactor container 12 to the expansion container 14. The filter may additionally prevent liquid from transferring from the compressible bioreactor container 12 to the expansion container 14.

As shown in FIG. 3, the interface plate 13 has a hollow ring-shaped protrusion 34 that extends from an upper side of the interface plate 13. An outer surface of the ring-shaped protrusion 34 connects to the lower part 20 of the compressible side wall 18 of the expansion container 14. An inner surface of the ring-shaped protrusion 34 has a plurality of apertures 21 spaced around the inner surface. The apertures 21 permit fluid communication between the compressible bioreactor container 12 and the expansion container 14. A plurality of walls 35 extend inwardly from the inner surface of the ring-shaped protrusion 34 toward the center of the interface plate 13. The walls 35 are positioned approximately at the center of each aperture 21. The walls 35 create a winding (non-linear) path for gas as it flows between the compressible bioreactor container 12 and the expansion container 14 and thereby increase the surface area that gas from the compressible bioreactor container 12 contacts as it flows into the expansion container 14. As the gas contacts the surfaces around the apertures 21, including the walls 35, water vapor in the gas condenses on these surfaces. The upper surface of the interface plate 13 within the inner surface of the ring-shaped protrusion 34 is sloped toward the apertures 21 to direct droplets of liquid back through the apertures 21 to return the liquid to the internal volume of the compressible bioreactor container. Accordingly, the increased surface area created by the walls 35 around the apertures 21 reduces the passage of liquid vapor out of the compressible bioreactor container 12 while providing apertures through which gas can freely flow between the compressible bioreactor container 12 and the expansion container 14.

The expansion container 14 has a bottom section formed by the interface plate 13, a top section 25 that is connected to a filter 19 having a gas outlet 26, and a compressible side wall 18.

A lower part 20 of the compressible side wall 18 is attached to the interface plate 13. The lower part 20 may include a rigid ring or similar for attaching to the interface plate 13. The compressible side wall 18 is compressible such that the top section 25 can move toward and away from the interface plate 13, varying the internal volume of the expansion container 14.

The compressible side wall 18 may be a bellows wall, having a concertina arrangement that allows the compressible side wall 18 to fold onto itself in order to compress. In particular, the compressible side wall 18 may comprise a series of alternately arranged inward folds 18a and outward folds 18b that allow the compressible side wall 18 to compress like a bellows or concertina. Rigid leaf portions extend between the inward folds 18a and the outward folds 18b. The inward folds 18a and outward folds 18b may be formed by thinned sections in the compressible side wall 18, with the inward folds 18a having a thinned section arranged on the outer surface of the compressible side wall 18, and the outward folds 18b having a thinned section arranged on the inner surface of the compressible side wall 18.

The expansion container 14 allows for the compressible bioreactor container 12 to expand and contract without greatly changing the pressure in the compressible bioreactor container 12. Alternatively or additionally, the expansion container 14 may be operable, for example, by being mechanically or manually compressed or expanded, to expand or retract the compressible side wall 18 of the expansion container 14 and thereby change a volume of the compressible bioreactor container 12. Alternatively or additionally, the expansion container 14 may be operable, for example, by being mechanically or manually compressed or expanded, to alter the pressure within the compressible bioreactor container 12.

As shown in FIGS. 1A and 1B, a cage 29 is provided around the expansion container 14 and keeps the expansion container 14 in line as it expands and contracts. The cage 29 comprises a first cage part 29a attached to the interface plate 13 and a second cage part 29b slidably mounted to the first cage part 29a. The second cage part 29b can slide in the direction of expansion and contraction of the expansion container 14. A block prevents the second cage part 29b from disengaging from the first cage part 29a.

Accordingly, the expansion container 14 can expand or contract depending on operation and environmental characteristics of the bioreactor 10. As the expansion container 14 expands and contracts, the cage 29 constrains movement of the expansion container 14 as the first and second cage parts 29a, 29b slide relative to each other.

The cage 29 also includes a clamp feature 30, in this example a lip. The clamp feature 30 is grippable by an actuator. In other examples where the bioreactor 10 does not include an expansion container 14, the lip 30 may be provided on the interface plate 13.

In some examples, a locking element may be provided on the first cage part 29a and/or the second cage part 29b to lock the second cage part 29b in a first contracted position (see FIG. 1A) and/or in a second expanded position (see FIG. 1B) with respect to the first cage part 29a. The locking element may be any suitable locking element, for example, a corresponding notch and protrusion. In other examples, the second cage part 29b may be held in the first contracted position and/or the second expanded position by an actuator.

As shown in FIGS. 2, 4 and 7A, the filter 19 has a hollow body that provides a channel through the filter 19. The channel is the gas outlet 26 of the bioreactor 10. The hollow body has a central portion 32, a first end 27 extending from a first side of the central portion 32 and a second end 28 extending from a second side of the central portion 32. The central portion 32 is wider than the first end 27 and the second end 28 to receive a filter element 33 therein. The filter element 33 extends across the channel such that all gas flow through the channel passes through the filter element 33. Any suitable filter element 33 that filters particulates, such as microbes or other contaminants, may be used. For example, the filter element 33 may be a polyethersulfone membrane or a polyvinylidene difluoride membrane. The first end 27 of the filter is positioned in an internal volume of the expansion container 14 and the second end 28 of the filter is positioned external to the expansion container 14. In some examples, the filter 19 may not comprise a second end 28 as illustrated.

The outlet 26 permits gas exchange between the internal volume of the expansion container 14 and an external environment, such as a bioreactor housing. In particular, as the pressure in the internal volume of the expansion container 14 increases, gas flows out of the expansion container 14 through the outlet 26. The pressure in the expansion container 14 may increase as a result of contraction of the expansion container 14 and/or contraction of the compressible bioreactor container 12, which forces gases from the compressible bioreactor container 12 into the expansion container 14. Furthermore, as the pressure in the internal volume of the expansion container 14 and/or the compressible bioreactor container 12 decreases, gas flows into the expansion container 14 through the outlet 26. The pressure in the expansion container 14 may decrease as a result of expansion of the expansion container 14 and/or expansion of the compressible bioreactor container 12, which draws gases from the expansion container 14 into the compressible bioreactor container 12.

In some embodiments, the outlet 26 may comprise a one-way valve to only permit gas to flow out of the bioreactor 10. A gas inlet may be provided in one of the compressible bioreactor container 12, the interface plate 13, or the expansion container 14. The gas inlet may comprise a one-way valve to only permit gas to flow into the bioreactor 10.

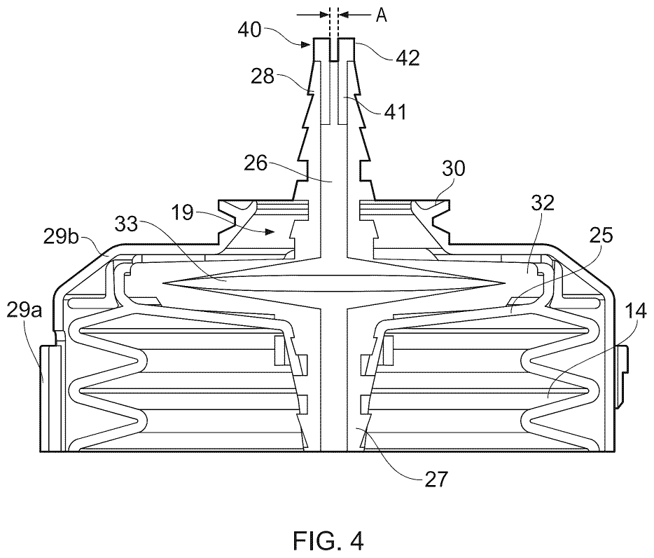

FIG. 4 shows a first example of a flow restrictor 40. The flow restrictor 40 is positioned in the second end 28 of the outlet 26. The flow restrictor 40 restricts the flow of gases through the outlet 26. This reduces the rate at which gas leaves the internal volume of the expansion container 14, thereby reducing the volume of gas that leaves the internal volume of the expansion container 14 during compression, and reducing the loss of liquid from the bioreactor 10 in the form of vapor. As gas flow out of the outlet 26 is restricted, this allows pressure to build up in the internal volume of the expansion container 14. Once the pressure in the expansion container reaches a certain level the expansion container 14 expands and receives the additional gas from the compressible bioreactor container 12.

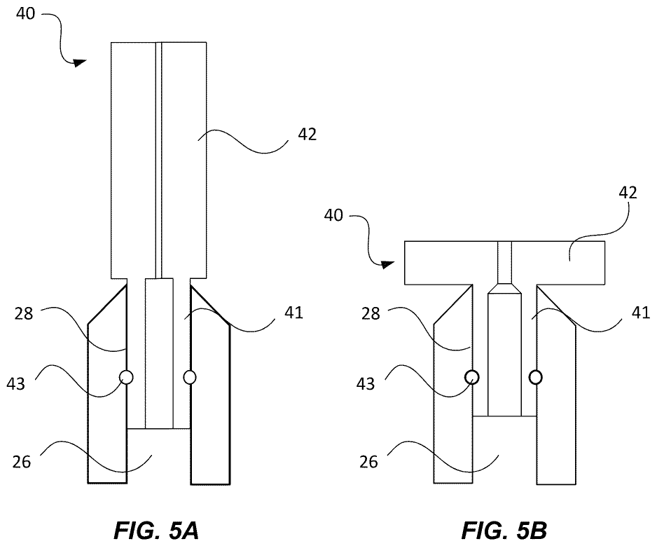

FIGS. 5A and 5B show examples of the flow restrictor 40 of FIG. 4. As shown in FIGS. 5A and 5B, the flow restrictor 40 has a body having a first end 41 and a second end 42. The body is hollow to provide a gas flow path that extends from the first end 41 to the second end 42. The inner diameter (A) of the hollow body is smaller than the diameter (B) of the outlet 26 (see FIG. 2). For example, the diameter (B) of the outlet 26 may be between about 3 mm and about 5 mm, for example, about 3.8 mm, and the inner diameter (A) of the hollow body may be between about 0.15 mm and about 1.5 mm, for example, about 0.5 mm or about 0.75 mm or about 1 mm. The flow restrictor 40 thereby restricts the diameter (B) of the outlet 26 to restrict gas flow out of the outlet. At the first end 41 of the body, an outer diameter of the body is the same or smaller than the diameter (B) of the outlet 26. This allows the first end 41 of the flow restrictor 40 to be inserted into the second end 28 of the outlet 26.

In some examples, as illustrated in FIGS. 5A and 5B, the first end 41 of the flow restrictor is inserted into the second end 28 of the outlet 26. The second end 42 of the body has a flange. The flange contacts an outer edge of the second end 28 of the outlet 26. The flange may work as a stopper to prevent the flow restrictor 40 from moving through the outlet 26. In one example, as shown in FIG. 4, the first end 41 of the flow restrictor 40 is press-fit into the second end 28 of the outlet 26. In another example, as shown in FIGS. 5A and 5B, the first end 41 comprises an O-ring 43 to provide a seal between the first end 41 of the flow restrictor 40 and the outlet 26. In another example, the flow restrictor may be integrally formed in the second end 28 of the outlet 26.

The inner diameter (A) of the hollow body may be between about 0.15 mm and about 1.5 mm. In some examples, the inner diameter (A) of the hollow body is between about 0.5 mm and about 1.0 mm. In some examples, the inner diameter (A) of the hollow body is about 0.50 mm, or about 0.75 mm, or about 1.00 mm.

As shown in FIGS. 5A and 5B, the second end 42 of the flow restrictor 40 may be different length. The length of the second end 42 of the flow restrictor 40 shown in FIG. 5A is greater than the length of the second end 42 of the flow restrictor 40 shown in FIG. 5B. In examples, the length of the second end 42 of the flow restrictor 40 may be between about 1 mm and about 15 mm. In examples, the length of the second end 42 of the flow restrictor 40 may be between about 1 mm and about 10 mm. In examples, the length of the second end 42 of the flow restrictor 40 is about 1.5 mm. In examples, the length of the second end 42 of the flow restrictor 40 is about 10 mm. It will be appreciated that the greater the length of the second end 42 of the flow restrictor 40 the more flow is restricted.

The flow restrictor 40 may be formed from any suitable material. The flow restrictor 40 may be formed from a gamma-irradiation resistant polymer. For example, the flow restrictor 40 may be formed from a polymer, such as a polyaryletherketone, for example, polyether ether ketone (PEEK). The flow restrictor 40 may be manufactured by machining (turning). Additionally or alternatively, the flow restrictor 40 may be manufactured by an additive process, or by molding.

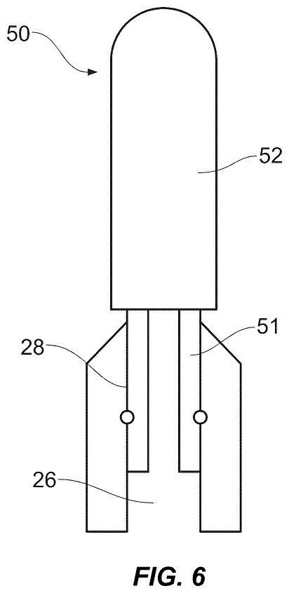

FIG. 6 shows a second example of a flow restrictor 50. The flow restrictor 50 is positioned in the second end 28 of the outlet 26 in the same way as the flow restrictor 40 shown in FIG. 4.

The flow restrictor 50 has a body having a first end 51 and a second end 52. The first end 51 of the body is hollow to permit gas to enter the flow restrictor 50. The second end 52 of the body has a porous material. In some examples, the second end 52 of the body may be formed from the porous material. In other examples, the flow restrictor 50 is the same as the flow restrictor described with reference to FIGS. 4 to 5B, and the second end 52 of the body is covered with a porous material.

The porous material permits restricted gas flow therethrough. This restricts gas flow out of the outlet 26.

The body of the flow restrictor 50 may be formed from any suitable material. The body of the flow restrictor 50 may be formed from a gamma-irradiation resistant polymer. For example, the body of the flow restrictor 50 may be formed from a polymer, such as a polyaryletherketone, for example, polyether ether ketone (PEEK). The body of the flow restrictor 50 may be manufactured by machining (turning). Additionally or alternatively, the body of the flow restrictor 50 may be manufactured by an additive process, or by molding. The porous material may be a sintered polymer. Any suitable polymer may be used, for example, polytetrafluoroethylene (PTFE), polyethylene (PE) or polypropylene (PP).

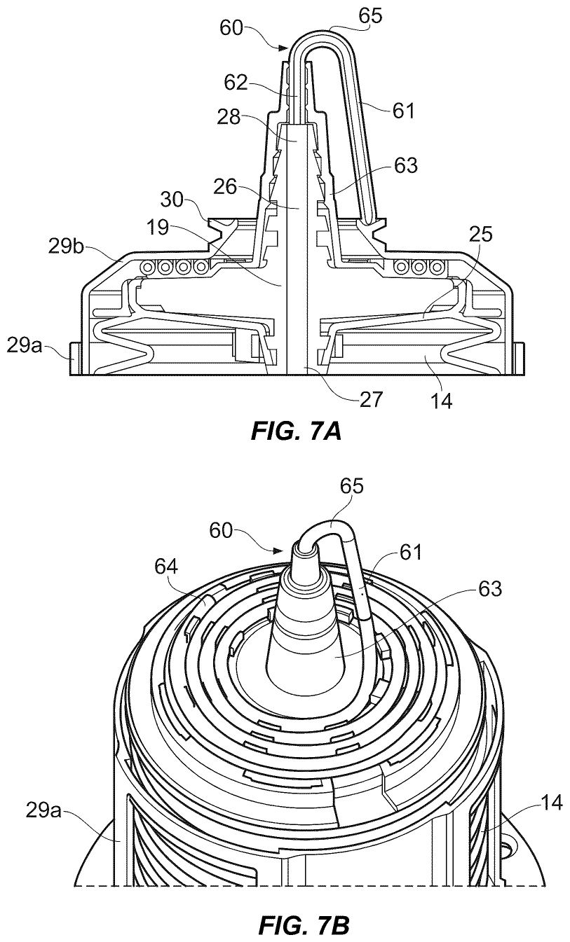

FIGS. 7A and 7B show a third example of a flow restrictor 60. The flow restrictor 60 has an elongate tube 61. A first end portion 62 of the elongate tube 61 is positioned in fluid communication with the second end 28 of the outlet 26. The elongate tube 61 increases the resistance to gas flow out of the outlet 26, thereby restricting gas flow through the outlet.

The elongate tube 61 is fixed in fluid communication with the outlet 26 by a tube support 63. The tube support 63 surrounds the second end 28 of the outlet 26 and the first end portion 62 of the elongate tube 61, and holds first end portion 62 of the elongate tube 61 in alignment with the second end 28 of the outlet 26 so as to permit fluid to flow therebetween.

The elongate tube 61 has an internal diameter that is smaller than the diameter (B) of the outlet 26. The internal diameter of the elongate tube 61 may be 0.25 mm to 1 mm. In some examples, the internal diameter the elongate tube 61 is 0.5 mm.

The length of the elongate tube 61 is 100 mm to 700 mm. In some examples, the length of the elongate tube 61 is 300 mm to 500 mm. In some examples, the length of the elongate tube 61 is 300 mm or 500 mm.

The elongate tube 61 has a bent portion 65 adjacent to the tube support 63. The bent portion 65 may be formed from a relatively rigid material in comparison with the material of the remainder of the tube, or the bent portion 65 may be reinforced, so as to prevent the bent portion from kinking and blocking gas flow.

As shown in FIG. 7A, the second cage part 29b covers the tube support 63 and at least a portion of the elongate tube including a second end portion 64 of the elongate tube.

As shown in FIG. 7B, the elongate tube 61 may be coiled to prevent kinks in the tube that will block gas flow and to reduce the footprint of the flow restrictor 60. The tube support 63 includes a circular base with guides for receiving the elongate tube 61 in a coiled arrangement.

According to the illustrated example, the elongate tube 61 is held fixed at the second end 28 of the outlet 26 by the tube support 63. In other examples, the first end portion 62 of the elongate tube 61 may be press-fit into the second end 28 of the outlet 26 or sealed in the second end 28 of the outlet 26 by an O-ring, such that the second end 28 retains the first end portion 62 of the elongate tube 61 in fluid communication with the outlet 26.

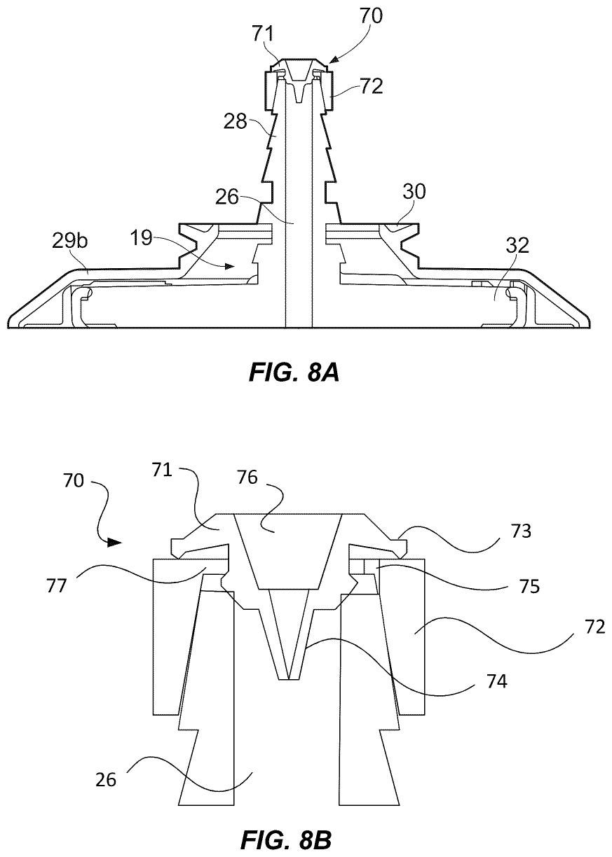

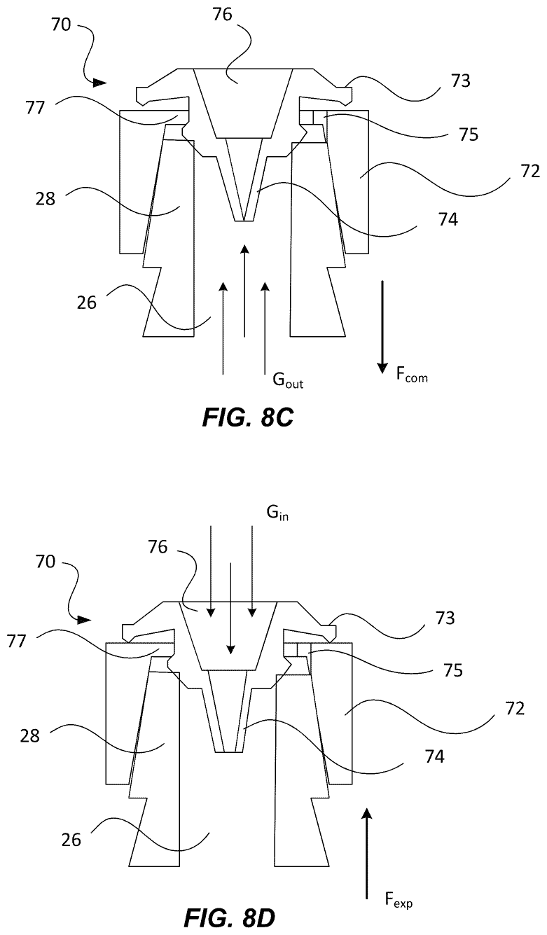

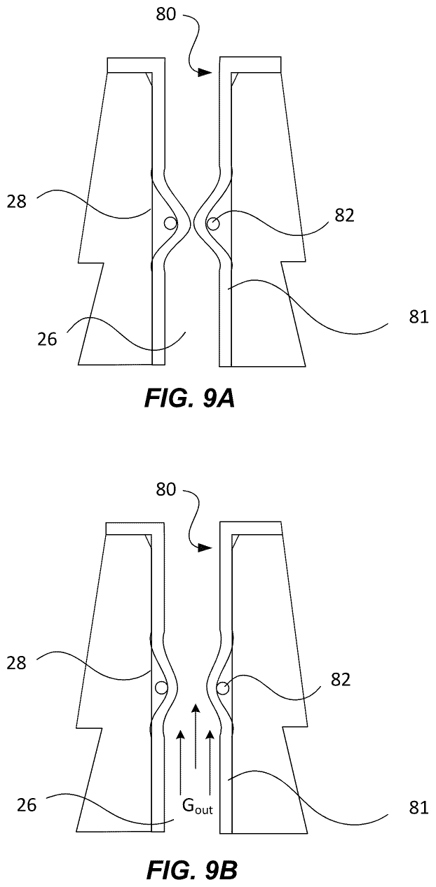

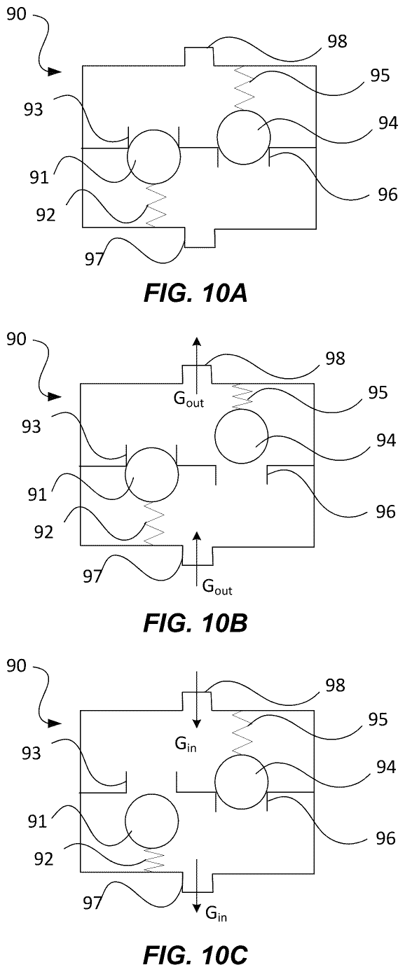

FIGS. 8A to 10C show examples of valve flow restrictors 70, 80, 90. FIGS. 8A to 8D show a fourth example of a flow restrictor 70 in the form of a combination umbrella and duckbill valve. FIGS. 9A and 9B show a fifth example of a flow restrictor 80 in the form of an elastic constriction valve. FIGS. 10A to 10C show a sixth example of a flow restrictor 90 in the form of a two-way ball valve. These valves 70, 80, 90 permit gas flow through the valve when the differential gas pressure at the gas outlet 26 is at or above a threshold differential pressure, and prevent gas flow when the differential gas pressure is below the threshold differential pressure. The valves 70, 80, 90 remain closed when the differential gas pressure is below the threshold differential pressure, thereby preventing the loss of fluids from the bioreactor container 12 in the form of vapor. However, the valves 70, 80, 90 open when the differential gas pressure at the gas outlet 26 is at or above a threshold differential pressure.

The valves 70, 80, 90 open as the differential gas pressure increases to a differential pressure at and above the threshold differential pressure. This provides a force profile at the valve 70, 80, 90, which increases at an approximately constant rate. In turn, this allows for the bioreactor container 12 to be expanded and/or contracted without significantly increasing force required to expand or contract the bioreactor container 12. This also prevents substantial fluctuations in pressure in the bioreactor container 12 and/or the expansion container 14 during expansion and/or contraction.

Other one-or two-way pressure-actuated valves (not shown) may be utilized in the present disclosure. The valve may be any valve that can be actuated by differential gas pressure in proximity to the valve. For example, the valve may be a membrane valve having one or more slits therein. As the differential gas pressure increases to a differential pressure at and above the threshold differential pressure, the membrane of the membrane valve flexes in the direction of relatively low gas pressure, opening the one or more slits and permitting gas to flow therethrough.

As shown in FIGS. 8A to 8D, the combination umbrella and duckbill valve 70 of the fourth example of the flow restrictor includes a valve element 71 and a support 72. As shown in FIG. 8B, the valve element 71 has an umbrella portion 73 that is a flange that extends away from a first, upper end of the valve element 71. The valve element 71 also has a duckbill portion 74 including elastomeric lips.

The support 72 has a valve seat 77 at an upper end of the support 72. The valve seat 77 has at least one opening 75 therein to permit gas flow therethrough. The valve seat may have a plurality of openings. In one example, the valve seat has four openings. The thickness of the valve seat may be selected so as to manipulate the differential gas pressure required to open the valve. In examples, the thickness of the valve seat is from approximately 0.6 to 1.2 mm. In some example, the thickness of the valve seat is approximately 0.6 mm, 0.8 mm, 1.0 mm, or 1.2 mm.

In a resting position, the umbrella portion 73 rests against the valve seat 77 blocks the opening(s) 75 in the valve seat 77. In a resting position, the elastomeric lips of the duckbill portion 74 rest against one another to block the flow of air through a central opening 76 of the valve element 71.

When the gas pressure in the gas outlet 26 greater than an external pressure such that the differential gas pressure is at or above the threshold differential pressure, as shown in FIG. 8C, the umbrella portion 73 lifts away from the support 72 to permit gas flow Gₒᵤₜ out of the bioreactor container 12 and/or the expansion container 14 through the opening 75 to flow out of the valve 70. The gas pressure in the gas outlet 26 may increase when the bioreactor container 12 and/or the expansion container 14 are compressed in the direction F_{com}, which increases the pressure within the bioreactor container 12 and/or the expansion container 14. When the differential gas pressure at the gas outlet 26 falls below the threshold differential pressure, the umbrella portion 73 returns to its resting position and rests against the upper end of the support 72, as shown in FIGS. 8B and 8D, to prevent the flow of gas through the opening 75.

When the gas pressure proximate to the central opening 76 of the valve element 71 is greater than the gas pressure in the gas outlet 26 such that the differential gas pressure is at or above the threshold differential pressure, as shown in FIG. 8D, the elastomeric lips of the duckbill portion 74 separate from one another to permit gas flow Gin into the bioreactor container 12 and/or the expansion container 14 through the central opening 76. The gas pressure proximate to the central opening 76 of the valve element 71 may increase relative to the gas pressure in the gas outlet 26 when the bioreactor container 12 and/or the expansion container 14 is expanded in the direction Fexp, which reduces the pressure within the bioreactor container 12 and/or the expansion container 14. When the differential gas pressure at the gas outlet 26 falls below the threshold differential pressure, the elastomeric lips of the duckbill portion 74 return to their resting position and rest against one another, as shown in FIGS. 8B and 8C, to prevent the flow of gas through the central opening 76.

The support 72 is press-fit onto an outer surface of the second end 28 of the outlet 26. According to an alternative example, the support 72 may be press-fit into the second end 28 of the outlet 26. In yet another example, the support 72 may be integrally formed with the second end 28 of the outlet 26.

As shown in FIGS. 9A and 9B, the elastic constriction valve 80 of the fifth example of the flow restrictor includes a resilient valve element 81 and a compression ring 82. The resilient valve element 81 is cylindrical and is formed of a resilient material. The resilient valve element 81 is biased toward its cylindrical shape. The compression ring 82 is provided about an outer surface of the resilient valve element 81. The compression ring 82 is formed from an elastic material. The compression ring 82 compresses a portion of the resilient valve element 81 in a resting position, so as to reduce the diameter of the resilient valve element 81 at this portion.

When the differential gas pressure at the gas outlet 26 is at or above the threshold differential pressure, as shown in FIG. 9B, compression ring 82 expands, expanding the diameter of the resilient valve element 81 to permit gas flow Gout out of, or gas flow into (not shown), the bioreactor container 12 and/or the expansion container 14 through the resilient valve element 81. The differential gas pressure at the gas outlet 26 may increase when the bioreactor container 12 and/or the expansion container 14 is compressed, which increases the pressure within the bioreactor container 12 and/or the expansion container 14 relative to an external pressure. The differential gas pressure at the gas outlet 26 may also increase when the bioreactor container 12 and/or the expansion container 14 is expanded, which decreases the pressure within the bioreactor container 12 and/or the expansion container 14 relative to the external pressure. When the differential gas pressure at the gas outlet 26 falls below the threshold differential pressure, the compressing ring returns to its resting position and reduces the diameter of the resilient valve element 81 at this portion, as shown in FIG. 9A, to prevent the flow of gas through the resilient valve element 81.

The elastic constriction valve 80 may be provided in an internal surface of the gas outlet 26. Alternatively, the elastic constriction valve 80 may be connected to an end of the gas outlet 26.

As shown in FIGS. 10A to 10C, the two-way ball valve 90 of the sixth example of the flow restrictor includes a first ball valve 91 connected to a first spring element 92, a first ball seat 93, a second ball valve 94 connected to a second spring element 95, and a second ball seat 96. The two-way ball valve 90 also has an outlet end 97 and an inlet end 98.

As shown in FIG. 10A, each of the first and second ball valves 91, 94, rest in their respective first and second ball seats 93, 96, in a resting position.

The two-way ball valve 90 may be connected to an end of the gas outlet (26, see FIG. 2). The two-way ball valve 90 may otherwise be incorporated into the gas outlet 26 by any suitable means.

When the gas pressure at the outlet end 97 (i.e., the gas pressure in the gas outlet 26) is greater than gas pressure at the inlet end 98 (i.e., an external pressure) such that the differential gas pressure is at or above the threshold differential pressure, as shown in FIG. 10B, the second ball valve 94 lifts away from the second ball seat 96 against the bias of the second spring element 95 to permit gas flow Gₒᵤₜ out of the bioreactor container 12 and/or the expansion container 14 through the valve 90. When the differential gas pressure at the gas outlet 26 falls below the threshold differential pressure, the second ball valve 94 is biased by the second spring element 95 to rest in the second ball seat, as shown in FIGS. 10A and 10C, to prevent the flow of gas through the valve 90.

When the gas pressure at the inlet end 98 (i.e., an external pressure) is greater than the gas pressure at the outlet end 97 (i.e., the gas pressure in the gas outlet 26) such that the differential gas pressure is at or above the threshold differential pressure, as shown in FIG. 10C, the first ball valve 91 lifts away from the first ball seat 93 against the bias of the first spring element 92 to permit gas flow Gin into the bioreactor container 12 and/or the expansion container 14 through the valve 90. When the differential gas pressure at the gas outlet 26 falls below the threshold differential pressure, the first ball valve 91 is biased by the first spring element 92 to rest in the first ball seat 93, as shown in FIGS. 10A and 10B, to prevent the flow of gas through the valve 90.

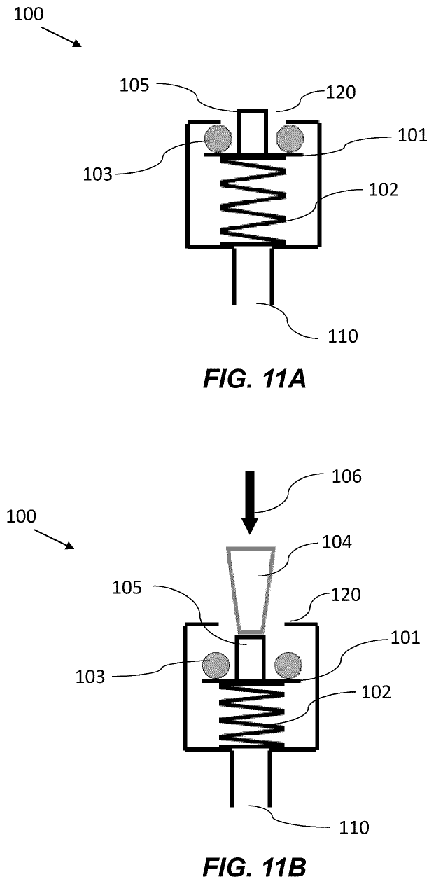

FIGS. 11A and 11B show a seventh example of a flow restrictor in the form of a user-actuatable valve 100. The user-actuatable valve 100 includes an outlet end 110 and an inlet end 120.

The user-actuatable valve 100 may be connected to an end of the gas outlet 26, (see FIG. 2). The user-actuatable valve 100 may otherwise be incorporated into the gas outlet 26 by any suitable means.

The user-actuatable valve 100 includes a movable plate 101 and a spring element 102. The movable plate 101 is provided with a seal 103 for sealing the movable plate 101 against the inlet end 120 preventing gas flow therethrough. In this example, the seal 103 is an O-ring with an internal diameter that exceeds a diameter of the inlet end 120. In other examples, the seal 103 may be omitted. For example, the movable plate 101 may comprise a flexible material operable to seal against a perimeter of the inlet end 120.

The movable plate 101 is operable to translate between a closed position and an open position, upon user actuation of the user-actuatable valve 100.

FIG. 11A shows the user-actuatable valve 100 when the movable plate 101 is in the closed position. As shown, when the movable plate 101 is in the closed position, the seal 103 is sandwiched between a peripheral wall of the inlet end 120 and the movable plate 101. The movable plate 101 is essentially impermeable to fluids so this configuration prevents gasses from leaving or entering the user-actuatable valve 100 through the inlet end 120.

The spring element 102 is operably connected to the movable plate 101 and arranged to bias the movable plate 101 into the closed position. The spring element 102 has a resilience selected so that differential pressure fluctuations during use of the bioreactor 10 do not inadvertently switch the movable plate 101 between the closed position and the open position.

The user-actuatable valve 100 also comprises a valve actuator for actuating the movable plate 101 between the closed position and the open position. In this example, the valve actuator comprises a control arm 104 operably connected to a controller (not shown). The movable plate 101 includes a connector 105 configured to selectively interface with the control arm 104.

The controller (not shown) is a button for depressing the control arm 104 toward the movable plate 101. The valve actuator is configured such that when a user actuates the button, the control arm 104 pushes to contact the connector 105, as shown by block arrow 106. A force of the control arm 104 overcomes a reactive force of the spring element 102 to separate the movable plate 101 from the inlet end 120. In this way, the valve actuator is configured to switch the movable plate 101 from the closed valve position to the open valve position.

In alternate examples, the user-actuatable valve 100 may be actuated by any other suitable valve actuator. For example, the user-actuatable valve 100 may form a twist valve, an electrically actuated valve (e.g., a solenoid valve) or a magnetically actuated valve.

FIG. 11B shows the user-actuatable valve 100 when the movable plate 101 is in the open position. In this position, the inlet end 120 is fluidically connected to the outlet end 110. This configuration permits gas flow out of, or gas flow into, the bioreactor container 12 and/or the expansion container 14 through the user-actuatable valve 100. When the movable plate 101 is in the open position, the pressure within the bioreactor container 12 and/or the expansion container 14 equilibrates relative to the external pressure at the gas outlet 26.

The controller may be configured such that further actuation of the controller by a user (e.g., releasing the button) switches the movable plate 101 from the open position to the closed position. Releasing the button lifts the control arm 104 away from the connector 105 of the movable plate 101. Without the force of the control arm 104 on the movable plate 101, the spring element 102 forces the movable plate 101 into the closed position. As such, the user-actuatable valve 100 is configured to enable users to switch the valve from a closed configuration (where the movable plate 101 is in the closed position) to an open configuration (where the movable plate 101 is in the open position) and vice versa.

The user may close the user-actuatable valve 100 during a compression mixing operation. This may reduce the quantity of water vapor lost from inside the bioreactor 10 during such operations. As detailed previously, during such compression mixing operations, the differential pressure may increase. After a compression mixing operation has completed, the user may open the user-actuatable valve 100 to relive pressure with the bioreactor 10. Such a ‘breathing operation’ may allow maximum air exchange with the contents of the bioreactor container 12 and/or the expansion container 14 and prevent a significant pressure differential from arising.

In contrast to the flow restrictors of FIGS. 8A to 10B that are actuated by a gas pressure differential, actuation of the user-actuatable valve 100 is independent from the gas pressure differential. The user-actuatable valve 100 may provide greater control over the environmental parameters experienced by the contents of the bioreactor container 12 and/or the expansion container 14.

In one example, the inlet end 120 of the user-actuatable valve 100 may be connected to an end of the gas outlet 26, while the outlet end 110 may be a free end at external pressure. Alternately, the outlet end 110 may be connected to an end of the gas outlet 26, while the inlet end 120 may be a free end at external pressure.

In an alternative embodiment, the valve actuator may be configured such that pressing the button switches the movable plate 101 from the open position to the closed position. For example, the control arm 104 may be weighted such that the weight of the control arm 104 on the movable plate 101 overcomes the spring force of the spring element 102. The controller may be configured such that pressing the button lifts the control arm 104 away from the connector 105, switching the movable plate 101 from the open position to the closed position. While the controller of the user-actuatable valve 100 is described as a button, any suitable controller may instead be used such as a lever, dial or electronic controller. The user-actuatable valve 100 described herein may be provided in the bioreactor 10 instead of, or in addition to, the flow restrictor 40, 50 or 60 or the valve 70, 80 or 90 of the preceding embodiments.

The use of the bioreactor 10 of the present disclosure is described with reference to FIGS. 1 to 10B. In use, the bottom wall 15 of the compressible bioreactor container 12 is tilted and/or moved in an axial direction of the compressible bioreactor container 12 by an actuator. This movement of the bottom wall 15 compresses and/or expands the compressible bioreactor container 12. This is done to mix or agitate the fluid in the internal volume of the compressible bioreactor container 12, or to control the volume of the compressible bioreactor container 12 prior to adding material into the compressible bioreactor container 12 or extracting a sample from the compressible bioreactor container 12, or to perform one or more cell processing steps.

As the compressible bioreactor container 12 is compressed, the pressure in the compressible bioreactor container 12 increases, thereby forcing gas to flow from the compressible bioreactor container 12 to the expansion container 14 to equalize the pressure within the compressible bioreactor container 12 and the expansion container 14. The flow restrictor 40, 50 or 60 or the valve 70, 80, 90 or 100 according to any of the embodiments described above prevents the free flow of gas out of the expansion container 14. As the gas flow out of the expansion container 14 is restricted, the expansion container 14 will expand to compensate for the increased pressure from the introduced gas. Accordingly, the flow restrictor 40, 50, 60 causes expansion of the expansion container 14 and reduces gas egress through the gas outlet 26, thereby reducing vapor loss through the gas outlet 26. Likewise, the valve 70, 80, 90, 100 blocks the flow of gas out of the expansion container when the valve 70, 80, 90, 100 is closed, thereby causing expansion of the expansion container 14 preventing gas egress, and vapor loss, through the gas outlet 26.

As the compressible bioreactor container 12 is expanded, the pressure in the compressible bioreactor container 12 decreases, thereby drawing gas from the expansion container 14 back into the compressible bioreactor container 12 to equalize the pressure within the compressible bioreactor container 12 and the expansion container 14. The flow restrictor 40, 50 or 60 or the valve 70, 80, 90 or 100 according to any of the embodiments described above prevents the free flow of gas from outside of the bioreactor 10 into the expansion container 14. As the gas flow into the expansion container 14 is restricted, the expansion container 14 will contract to compensate for the reduced pressure due to the movement of gas out of the expansion container 14.

The expansion container 14 may also be locked or held in a contracted position by the cage 29 to permit gas exchange in the bioreactor 10. The expansion container 14 is first actuated to a contracted position by actuating the second cage part 29b to the first contracted position with respect to the first cage part 29a as shown in FIG. 1A. The second cage part 29b is locked or held in place with respect to the first cage part 29a to prevent expansion of the expansion container 14. The bottom wall 15 of the compressible bioreactor container 12 is moved upwards, toward the expansion container 14 by the actuator to compress the compressible bioreactor container 12, thereby increasing the gas pressure at the outlet 26 and expelling gasses from a headspace above the fluid in the internal volume of the bioreactor container 12, through the contracted expansion container 14 and the outlet 26. The bottom wall 15 of the compressible bioreactor container 12 is then moved downwards, away from the expansion container 14, by the actuator to expand the compressible bioreactor container 12, thereby reducing the gas pressure at the outlet 26 and drawing ambient gas into the internal volume of the bioreactor compressible bioreactor container 12 through the outlet 26 and the contracted expansion container 14. The second cage part 29b can then be released from the first contracted position such that the second cage part 29b can freely move with respect to the first cage part 29a and the expansion container 14 can freely expand and contract.

### Pressure Test Data

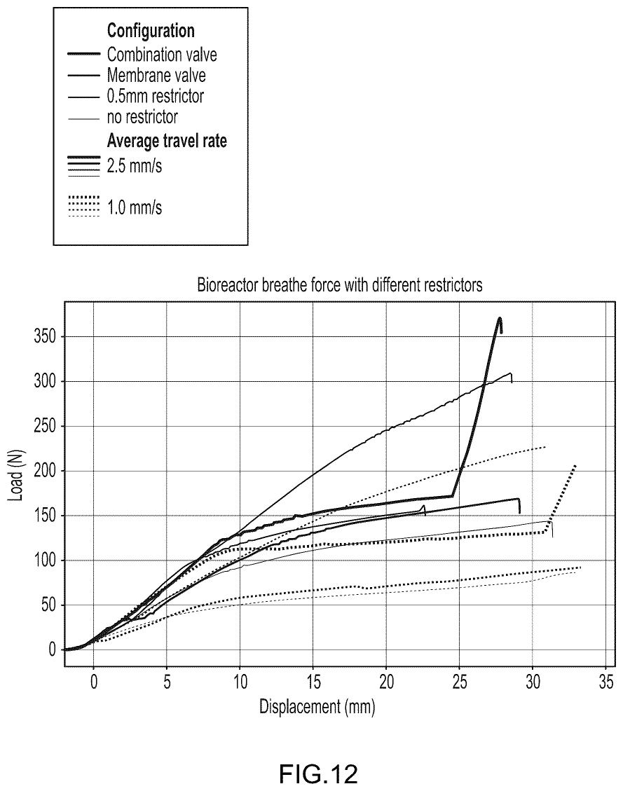

FIG. 12 shows the results of compression test data for three flow restrictors, a combination umbrella and duckbill valve (combination valve) with a valve seat of 0.8 mm thickness, a membrane valve with two slits therein (i.e., cross-shaped slits), a restrictor having a restricted diameter of 0.5 mm, and a control with no flow restrictor. The graph indicates the load required to compress the bioreactor container for each of the flow restrictors.

A first test was performed with a compression of the bioreactor container 12 at a speed of 2.5 mm/s as indicated by the solid line. A second test was performed with a compression of the bioreactor container 12 at a speed of 1.0 mm/s as indicated by the broken line. The expansion container 14 remained locked in a contracted position during this test.

The control with no flow restrictor requires the least force to compress the bioreactor container. The load increases at a relatively low rate as the displacement of the bioreactor increases.

The force required to compress the bioreactor container with the membrane valve is marginally higher than the force required for the control. The rate at which the load increases as the displacement of the bioreactor container increases is comparable with the rate of the control.

The force required to compress the bioreactor container with the combination valve is higher than that of the control. In one test, the force required to compress the combination valve is higher than the membrane valve at lower levels of displacement, and equivalent to the membrane valve at greater levels of displacement. In another test, the force required to compress the combination valve is higher than the membrane valve at all levels of displacement. The rate at which the load increases as the displacement of the bioreactor container increases is lower than the rate of the control and the membrane valve.

The force required to compress the combination valve is the highest. The rate at which the load increases as the displacement of the bioreactor container increases is also the highest.

Therefore, the membrane valve provides the lowest force required to compress the bioreactor container. This reduces the load on an actuator compressing the bioreactor container. The combination valve allows for the bioreactor to be compressed at a more steady rate as compared with the other flow restrictors. This allows for a more steady load to be applied by an actuator when compressing the bioreactor container, and also reduces the load required to compress the bioreactor container in comparison with the reduced diameter flow restrictor. The reduced diameter flow restrictor provides the highest force required to compress the bioreactor.

According to the illustrated examples, the filter is provided in the gas outlet 26 of the expansion container 14. In other examples, the filter may be provided in any suitable position between the flow restrictor 40, 50 or 60 or the valve 70, 80 or 90 and the fluid contained in the compressible bioreactor container 12 so as to prevent the introduction of contaminants into the fluid. For example, the filter may be provided in the flow restrictor 40, 50, 60, valve 70, 80 or 90, the filter may be provided in the interface plate 13 between the expansion container 14 and the compressible bioreactor container 12, or the filter may be provided in compressible bioreactor container 12 above the level of the fluid in use.

According to the illustrated examples, the outlet 26 is provided in the expansion container 14. In other examples, the bioreactor does not comprise an expansion container and the outlet 26 is provided in the compressible bioreactor container 12, for example, in the interface plate 13.

Generally, it will be appreciated by persons skilled in the art that the above embodiments have been described by way of an example only and not in any limitative sense, and that various alternations and modifications are possible without departing from the scope of the invention as defined by the appended claims. Various modifications to the detailed designs as described above are possible, for example, variations may exist in shape, size, arrangement, assembly, sequence or the like. For example, any one of the enclosures, planar interfaces, component retaining elements or the like may be used in any suitable combination. Moreover, while the present disclosure has been described in relation to an automated process, it will be appreciated by persons skilled in the art that a user may manually, or semi-automatedly, undertake one or more of the above process steps.

1. A bioreactor comprising:
a compressible bioreactor container configured to hold a cell suspension in an internal volume of the compressible bioreactor container, the compressible bioreactor container comprising a gas outlet in fluid communication with the internal volume of the compressible bioreactor container and configured to permit gas flow out of the internal volume of the compressible bioreactor container in use;
wherein the gas outlet comprises a flow restrictor configured such that when the compressible bioreactor container is compressed in use the flow restrictor restricts gas flow out of the gas outlet.

2. The bioreactor according to claim 1, wherein the bioreactor further comprises an expandable expansion container having an internal volume fluidly connected to the internal volume of the compressible bioreactor container.

3. The bioreactor according to claim 2, wherein the expansion container comprises the gas outlet.

4. The bioreactor according to claim 2, wherein the compressible bioreactor container comprises a base section, a top section, and a compressible side wall extending between the base section and the top section, and wherein the expansion container is connected to the top section of the compressible bioreactor container.

5. The bioreactor according to claim 4, wherein the top section of the compressible bioreactor container comprises an interface plate having an opening, and wherein the expansion container is mounted to the interface plate at the opening such that the internal volume of the expansion container is fluidly connected to the internal volume of the compressible bioreactor container via the opening in the interface plate.

6. The bioreactor according to claim 5, wherein the expansion container comprises a base section, a top section, and a compressible side wall extending between the base section and the top section, and wherein the base section is attached to the interface plate.

7. The bioreactor according to claim 5, wherein the interface plate includes one or more ports formed therein.

8. The bioreactor according to claim 1, wherein the flow restrictor comprises a valve.

9. The bioreactor according to claim 8, wherein the valve includes a user-actuatable valve and actuation of the valve is independent of a differential gas pressure across the valve.

10. The bioreactor according to claim 8, wherein the valve permits at least one flow chosen from among gas flow into the gas outlet and gas flow out of the gas outlet when a differential gas pressure at the gas outlet is at or above a threshold differential pressure.

11. The bioreactor according to claim 10, wherein the valve is a two-way valve.

12. The bioreactor according to claim 11, wherein the valve comprises a combination of an umbrella valve and a duckbill valve.

13. The bioreactor according to claim 1, wherein the flow restrictor comprises a flow restriction path having a restricted diameter.

14. The bioreactor according to claim 13, wherein the restricted diameter is between about 0.15 mm and about 1.5 mm.

15. The bioreactor according to claim 14, wherein the restricted diameter is between about 0.5 mm and about 1.0 mm.

16. The bioreactor according to claim 13, wherein the gas outlet has an outlet diameter, and wherein the restricted diameter is smaller than the outlet diameter.

17. The bioreactor according to claim 1, wherein the flow restrictor comprises a porous structure arranged to restrict the gas flow out of the outlet.

18. The bioreactor according to claim 17, wherein the porous structure is a sintered material.

19. The bioreactor according to claim 1, wherein the flow restrictor is disposed in the gas outlet via a press-fit into the gas outlet.

20. The bioreactor according to claim 1, wherein the flow restrictor is disposed over the gas outlet via a press-fit onto an outer surface of the gas outlet.

21. The bioreactor according to claim 1, wherein the flow restrictor is integrally formed with the gas outlet.

22. The bioreactor according to claim 1, wherein flow restrictor comprises a tube fluidly connected to the gas outlet.

23. The bioreactor according to claim 22, wherein an internal diameter of the tube is between about 0.2 mm and about 1 mm.

24. The bioreactor according to claim 23, wherein the internal diameter of the tube is about 0.5 mm.

25. The bioreactor according to claim 1, wherein a length of the tube is at least about 100 mm.

26. The bioreactor according to claim 25, wherein the length of the tube is between about 300 mm and about 500 mm.

27. The bioreactor according to claim 1, further comprising a filter provided in a gas flow path between the internal volume of the compressible bioreactor container and at least one component chosen from among the gas outlet and the flow restrictor.

28. The bioreactor according to claim 27, wherein the filter is provided in the gas outlet.

29. The bioreactor according to claim 1, further comprising one or more ports configured for at least one material transfer chosen from among introducing material into an internal volume of the compressible bioreactor container and removing material from the internal volume of the compressible bioreactor container.

30. A method of mixing a cell suspension in a bioreactor, the method comprising:
providing a bioreactor comprising a compressible bioreactor container with a gas outlet comprising a flow restrictor;
introducing a cell suspension to an internal volume of the compressible bioreactor container;
compressing the compressible bioreactor container to reduce the internal volume of the compressible bioreactor container; and
while compressing the compressible bioreactor container, restricting gas flow through the gas outlet by the flow restrictor.

31. The method according to claim 30, wherein the compressible bioreactor container comprises an expansion container having an internal volume fluidly connected to the internal volume of the compressible bioreactor container, and wherein the method comprises displacing gas from the internal volume of the compressible bioreactor container into the internal volume of the expansion container during compression of the compressible bioreactor container.

32. The method according to claim 30, wherein the flow restrictor comprises a valve, and wherein restricting the gas flow through the gas outlet by the flow restrictor comprises blocking gas flow through the valve when a differential gas pressure at the gas outlet is below a threshold differential pressure and permitting gas flow through the valve when the differential gas pressure at the gas outlet is at or above the threshold differential pressure.
